(19) United States
(12) Patent Application Publication
(10) Pub. No.: US 2003/0036678 A1
(43) Pub. Date: Feb. 20, 2003

(54) Method and apparatus for remote sexual contact

(76) Inventor: Touraj Abbassi, Baku (AZ)

Correspondence Address: Jack I. J'maev, 12616 Lewis Ave, Chino, CA, 91710, US

(21) Appl. No.: 09/929,645

(22) Filed: Aug. 14, 2001

(51) Int. Cl. A61F 5/00; H04B 3/36

(52) U.S. Cl. 600/38; 340/407.1

(57) ABSTRACT

Remote sexual intercourse is enabled using mechanical surrogates in the form of human genital components. Pressure and displacement sensors perceive action upon the surrogate. Actuators for pressure and displacement enable a second surrogate to mimic the contact imparted on the first. A computer program repeats contact messages comprising contact characteristics that describe the nature of a physical contact perceived by a surrogate with full duplex capability.

### BACKGROUND OF THE INVENTION

1. Technical Field

This invention pertains to the field of remote physical contact, and specifically to remote sexual contact.

2. Description of the Prior Art

Human contact has always involved stimulation of other people's senses, be it at an informal level or in a more intimate context. The need for sensory perception during the course of human contact can be driven by a myriad of motives. In one context, human emotional involvement has always led to an affectionate caress of a potential or current mate.

Men and women hold hands, kiss and engage in more prurient appealing activities. When humans desire more intimate contact, they inevitably engage in sexual intercourse. The applicant has created other apparatus that exploit communication networks to enable people to exchange a kiss while being separated by vast distances. That apparatus relies on a method of remote physical contact and is described in the applicants co-pending application entitled “Method and Apparatus for Remote Physical Contact”, Application No. 60/296,907, which is incorporated herein by reference in its entirety.

In the referenced application, the applicant teaches a means to enable remote physical contact between people. The method described in the referenced application is based on the exchange of stimulus information from one mechanical surrogate to another. The apparatus described in the Method and Apparatus for Remote Physical Contact is a mechanical surrogate in the form of a human lip. The human lip mechanical surrogate comprises sensors and actuators that enable one surrogate to sense a kiss. The attributes of the kiss, as defined by the sensor readings, are conveyed to a remote location where a second surrogate recreates the kiss so that a second user can experience the kiss imparted onto the first lip surrogate.

Because humans also desire contact that is more intimate than a kiss, new mechanical surrogates must be realized so that humans can engage in sexual intercourse while being separately located. These surrogates can be fashioned in the form of human genitalia.

What is lacking in the known art is a mechanical surrogate that transmits tactile or other physical stimulus from one human user to another to enable remote sexual intercourse. The present invention does just this; it enables people to expand on the notion of teleconferencing or computer communications by adding a capability to engage in intimate human contact.

### SUMMARY OF THE INVENTION

The present invention comprises a method for remote sexual contact that is based on the use of a female surrogate that accepts physical contact from a user. The method provides for monitoring the physical contact imparted onto the female surrogate through the use of sensors. Contact messages are formed that depict the nature of the physical contact. The contact messages are then conveyed to a male surrogate that is capable of mimicking the physical contact experienced by the female surrogate.

In order to realize the method, the present invention further comprises a system that employs the method, a male surrogate apparatus and a female surrogate apparatus. In the system, the female surrogate has a vaginal cavity that can be used by a user. The female surrogate creates contact messages that are indicative of the physical contact imparted upon the vaginal cavity. The female surrogate is connected to a first user computer that receives the contact messages and then conveys these to a communications network. A second user computer receives the contact messages from the communications network and then conveys these to a male surrogate. The male surrogate comprises an artificial penis mounted in a housing that can be extended from the housing in accordance with contact messages it receives.

The present invention further comprises a female surrogate. In one embodiment of the female surrogate, a vaginal cavity having an inner surface is provided. Sensors are disposed around the perimeter of the vaginal cavity and enable the female surrogate to detect the amount of penetration of an object into the cavity. The female surrogate further comprises an electronics unit that creates contact messages according to the readings perceived by the sensors. These contact messages are then conveyed to external devices. The sensors in the female surrogate can be either pressure sensors or strain gages.

In another embodiment of the female surrogate, constriction actuators are disposed around the circumference of the vaginal cavity. The electronics unit receives contact messages from an external device. These contact messages indicate the amount of force that must be exerted upon an object inserted in the vaginal cavity. The electronics unit commands the constriction actuators to reduce the diameter of the vaginal cavity so that the appropriate amount of force results on an object inserted therein.

One form of a constriction actuator can be realized using a constriction ring with two ends that are fed through slots in a pressure block. The ends of the constriction ring are then affixed to a stop block. A linear actuator can then be used to push upon the pressure block causing the two end of the constriction ring to be pulled through the slots in the pressure block. This causes the constriction rings circumference to be reduced resulting in a reduced diameter in the vaginal cavity.

In order to be able to sense when a male user has ejaculated, the female surrogate may further comprise a moisture sensor disposed in the vaginal cavity. The electronics unit in this alternative embodiment is capable of detecting when the moisture sensor is activated. In response, the electronics unit will create a contact message that indicates the presence of moisture in the female surrogates vaginal cavity.

The present invention further comprises a male surrogate that comprises a housing with an artificial penis that protrudes therefrom. The artificial penis has an external surface and a tip. Housed in the housing is an extension mechanism that causes the artificial penis to be moved outward from the housing. The amount of protrusion from the housing is determined by an electronics unit and is based on contact messages received from an external device. The electronics unit receives contact messages by means of an interface. The extension mechanism itself can be built using a heavy gage wire attached to a pulley at one end and to the artificial penis at the other. A stepper or servo motor is used to drive the pulley. As the pulley operates, the heavy gage wire is wound onto the pulley.

In another embodiment of the present invention, the male surrogate comprises a plurality of sensors disposed about the outer surface of the artificial penis. These sensors monitor the amount of pressure that the artificial penis is exposed to. These pressure readings are then used as a basis of contact messages that the electronics unit creates and forwards to other devices using an interface.

In yet another embodiment of the male surrogate, a flexible sheath is disposed around the artificial penis. The sheath forms an expansion cavity that accepts a thickened fluid. A pump is used to force the thickened fluid from a reservoir into the expansion cavity. This causes the overall diameter of the artificial penis to increase. The amount of fluid pumped into the expansion cavity is based on contact messages received by the electronics unit and are used to mimic the amount of force experienced by female surrogate.

Additional embodiments of the male surrogate comprise an ejaculant system. The ejaculant system comprises a reservoir for containing a quantity of ejaculant fluid. This fluid is forced by a pump through a catheter ending in the tip of the artificial penis. The pump is commanded to dispense some amount of ejaculant fluid in accordance with a contact message.

### BRIEF DESCRIPTION OF THE DRAWINGS

The foregoing aspects are better understood from the following detailed description of one embodiment of the invention with reference to the drawings, in which:

FIG. 1 is a block diagram that depicts the use of male and female surrogates in a networked environment;

FIG. 2 is a pictorial representation of a male surrogate according to the present invention;

FIG. 3A is a pictorial representation of the male surrogate in a retracted state;

FIG. 3B is a pictorial representation of the male surrogate in an extended state;

FIG. 4 is pictorial representation of a suite of sensors integral to a male surrogate;

FIG. 5 is a pictorial representation of a penis expansion system according to the present invention;

FIG. 6A is a pictorial representation in cross section of an artificial penis 75;

FIG. 6B is a pictorial in cross section of an expanded artificial penis;

FIG. 7 is a pictorial representation of an ejaculant system according to the present invention;

FIG. 8 is a pictorial representation of a female surrogate sensor system;

FIG. 9 is a pictorial diagram that depicts the placement of constriction actuators within the female surrogate; and

FIG. 10 is a detailed view of a constriction actuator according to the present invention.

### DETAILED DESCRIPTION OF THE INVENTION

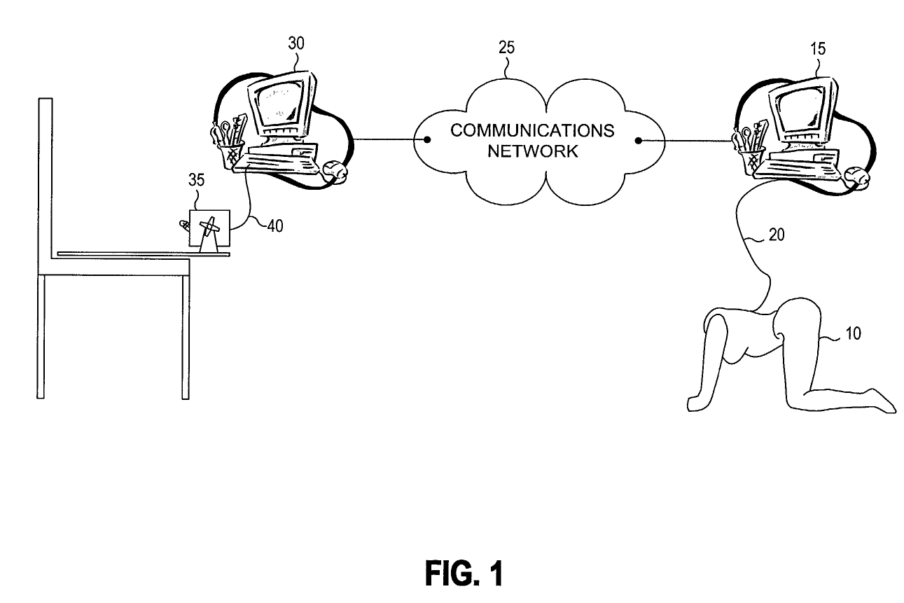

FIG. 1 is a block diagram that depicts the use of male and female surrogates in a networked environment. A female surrogate 10 interfaces to a first user computer 15 by means of a first serial interface 20. The first user computer 15 interfaces to a communications network 25. The communications network 25 allows the first computer 15 to communicate with a second user computer 30. The second user computer 30 interfaces to a male surrogate 35 also by means of a second serial interface 40. Although the interfaces used to connect the surrogates to the user computers are described as serial interfaces, the invention should not be limited to only serial interfaces. Any suitable interface type can be used. It should be noted that the communications network may be any suitable networking structure such as the Internet. Also, the communications network may be comprised of a simple serial interface connecting the first user computer 15 to the second user computer 30.

In operation, a male user utilizes the female surrogate 10. The female surrogate 10 senses penetration of the male user's penis and creates contact messages that indicate, among other factors, the depth of the penetration. As the male continues to use the female surrogate 10, additional contact messages are created on a continuous basis to reflect a real-time state of the contact between the male user and the female surrogate 10. Contact messages emanating from the female surrogate 10 and are propagated to the first user computer 15 by way of the serial interface 20. Communications software executing in the first user computer 15 dispatches the contact messages from the female surrogate 10 to the second user computer 30 using the communications network 25.

As contact messages arrive at the second user computer 30, they are conveyed to a male surrogate 35 by means of the second serial interface 40. When the male surrogate 35 receives the contact messages from the second user computer 30, the male surrogate 35 replicates the activity of the male human user imparted onto the female surrogate 10. A female user can then position herself in front of the male surrogate 35 in order to experience the activities of the male human user.

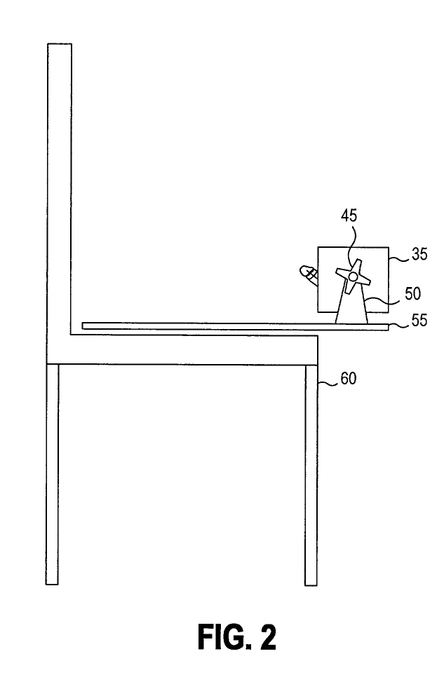

FIG. 2 is a pictorial representation of a male surrogate according to the present invention. The male surrogate 35 comprises a housing that encloses several electronic and mechanical components that collectively enable the male surrogate 35 to mimic the penetration of a penis. The male surrogate 35 may also comprise a mounting structure comprising adjustment knob 45 that allows the male surrogate housing to attach pivotally to a support bracket 50. The support bracket 50 is itself mounted to a seat platform 55. The seat platform 55 allows a female user to use the male surrogate 35 while seated in any ordinary chair 60. The seat platform 55 will ordinarily be upholstered on the top and possibly the bottom surface to provide for additional comfort during use.

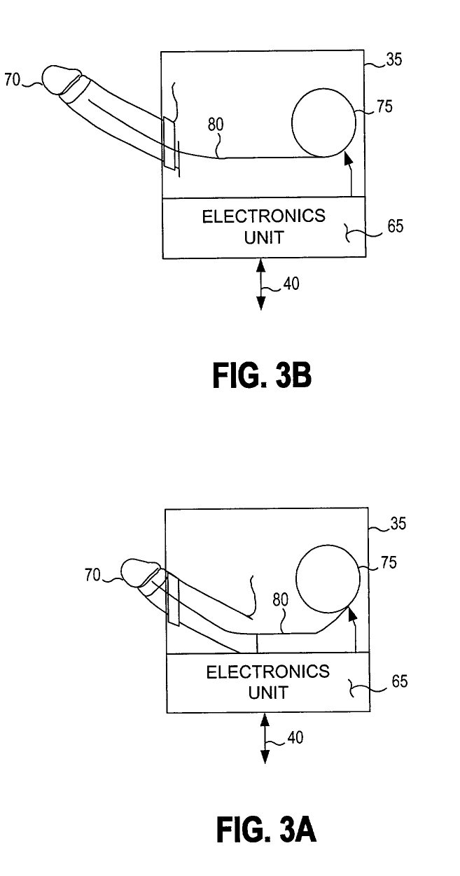

FIG. 3A is a pictorial representation of the male surrogate in a retracted state. The male surrogate 35 comprises a electronics unit 65 that receives contact messages by means of a serial interface 40. The contact messages convey the amount of penetration the artificial penis 70 must achieve. Contact messages arriving in real-time by serial interface 40 convey a sequence of penetration states. In response to each contact message, the electronics unit 65 will command an extension mechanism 75 to extend the artificial penis 70.

FIG. 3B is a pictorial representation of the male surrogate in an extended state. The extension mechanism 75 comprises either a stepper motor or a servo driving a segment a heavy gauge wire 80. The heavy gauge wire is normally spooled in the confines of the extension mechanism 75. As the electronics unit 65 commands the extension mechanism 75 to extend the artificial penis 70, the heavy gauge wire 80 is fed outward causing the artificial penis 70 penetrate a female user. It should be noted that the extension mechanism 75 is much akin to extension mechanisms used to raise automobile antennas. The extension mechanism 75 further comprises position sensors that enable the electronics unit 65 to determine the amount of extension realized that any given moment. It should be noted that the heavy gauge wire need not be metallic. Any pliable material, such as fiberglass or plastic can be used. Again, the type of wire used should be construed as limiting the scope of the invention.

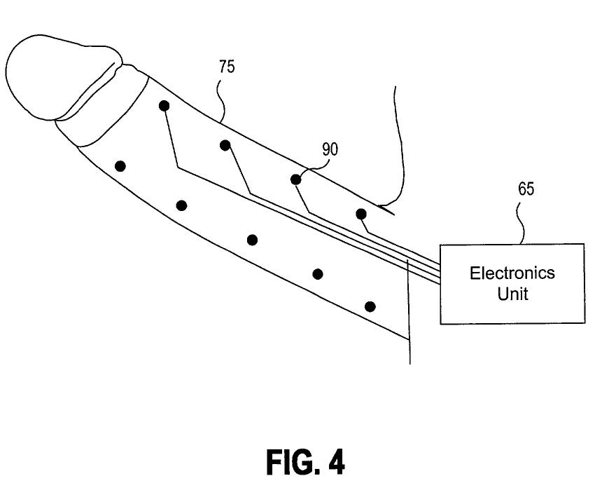

FIG. 4 is pictorial representation of a suite of sensors integral to a male surrogate. The male surrogate 35 may further comprise a plurality of sensors that enable the male surrogate 35 to perceive pressure whenever it is inserted into a cavity. Hence, when a female user exerts pressure onto the male surrogate 35, the sensors will register a pressure change. The electronics unit 65 monitors the status of the sensors and incorporates changes in pressure into contact messages. The contact messages are then conveyed to the second user computer 30 using a serial interface 40. Software executing in the second user computer 30 then transmits the contact messages to the first user computer 15 by means of a communications network 25.

The first user computer then conveys the contact message to the female surrogate 10 using a serial interface 20. Actuators in the female surrogate 10 then replicate the pressure that the male surrogate experiences so that the male user will sense the pressure imparted by the female user upon the male surrogate. This is described in greater detail infra.

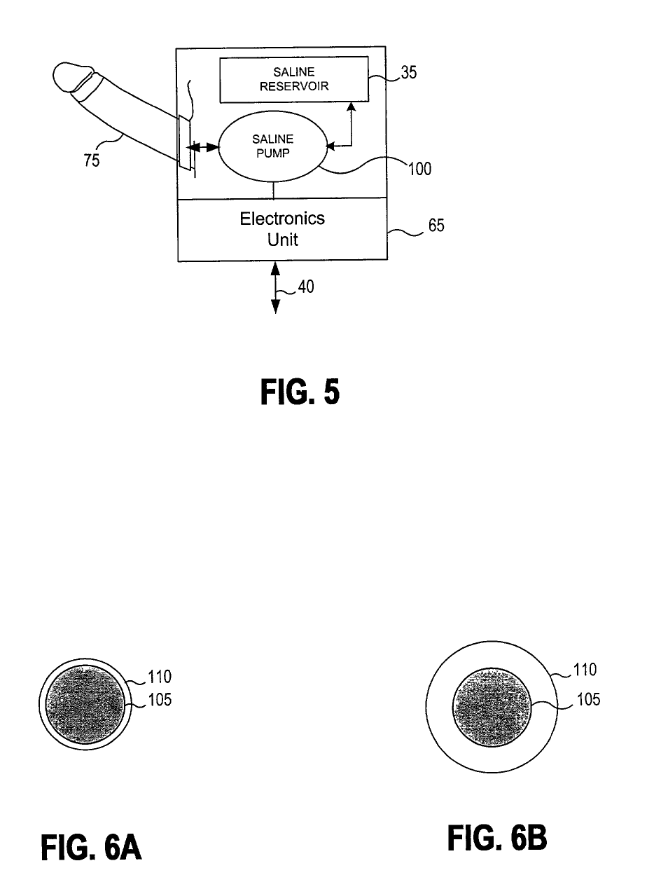

FIG. 5 is a pictorial representation of a penis expansion system according to the present invention. The male surrogate 35 may further comprise a system for expanding the diameter of the artificial penis 75. A thickened saline solution is contained in a saline reservoir 95. The saline is pumped into the artificial penis 75 by a saline pump 100. It should be noted that the expansion system can be used with any suitable expansion liquid—a thickened saline solution is only one example and is not intended to limit the scope of the invention described here.

FIG. 6A is a pictorial representation in cross section of an artificial penis 75. The core 105 of the artificial penis 75 is comprised of a pliable foam material such as styrene. It should be noted that the invention is not limited to embodiments using only styrene. Surrounding the penis core 105 is a pliable sheath 110. The pliable sheath 110 forms a cavity that accepts saline solution from the saline pump 100.

FIG. 6B is a pictorial in cross section of an expanded artificial penis. As the saline pump 100 pumps saline from the reservoir 95, the cavity formed by the sheath 100 expands. This cavity is called the “expansion cavity”. In the preferred embodiment, a thickened saline solution is used as a working fluid. However, any suitable fluid can be used as an expansion fluid and the use of a thickened saline solution is not intended to limit the scope of the present invention.

As the male user begins using the female surrogate 10, sensors in the female surrogate 10 will detect the amount of pressure exerted by the male user upon the female surrogates vaginal cavity (see infra). This pressure will be replicated by the male surrogate by expanding the artificial penis 75. The electronics unit 65 in the male surrogate will command the saline pump 100 to pump the saline into the cavity formed between the core 105 of the artificial penis 75 and the sheath 110 surrounding the core 105

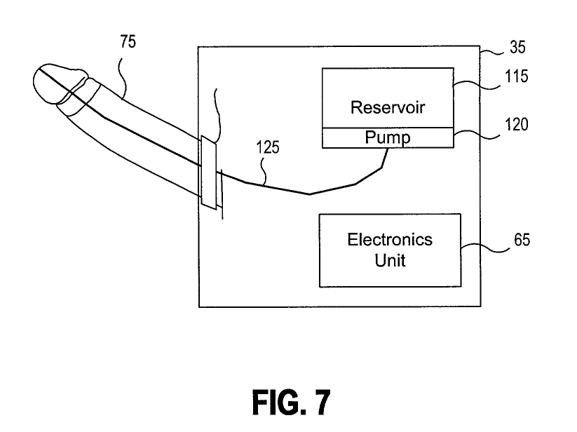

FIG. 7 is a pictorial representation of an ejaculant system according to the present invention. The male surrogate 35 may further comprise an ejaculant system. The ejaculant system comprises a fluid reservoir 115 for containing a quantity of ejaculant fluid, typically but not limited to artificial semen. The artificial semen is pumped through a semen pump 120. The output of the semen pump is directed to a catheter 125 that terminates at the tip of the artificial penis 75.

Upon receiving an appropriate contact message from the second user computer 30, the electronics unit 65 will command the semen pump 120 to dispense approximately 15 milliliters of the fluid stored in the semen reservoir 115.

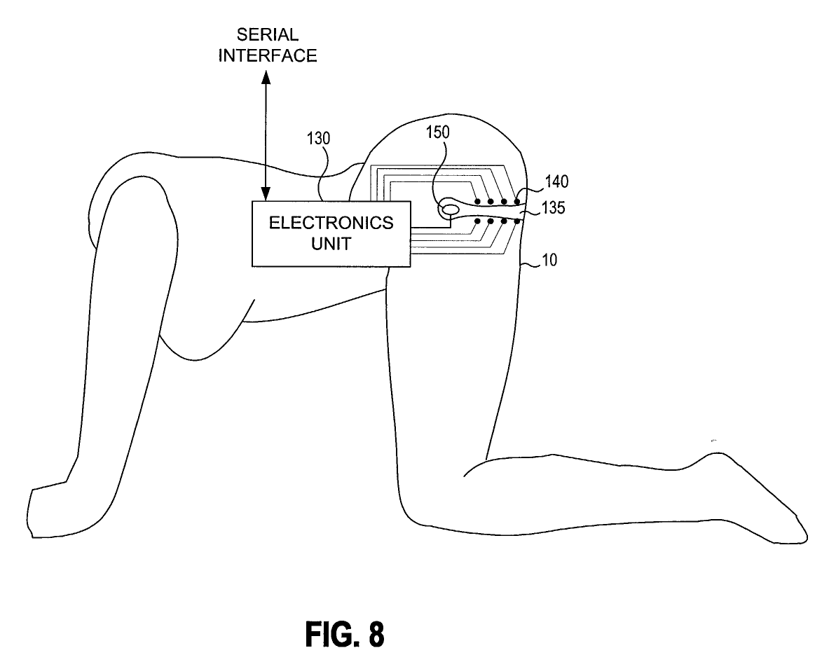

FIG. 8 is a pictorial representation of a female surrogate sensor system. The female surrogate 10 comprises a plurality of sensors 140 mounted on the periphery of a vaginal cavity 135. As a male user uses the female surrogate 10, sensors detect the depth of penetration of the user into the vaginal cavity 135. The sensors 140 can be either pressure sensors or strain gages.

The female surrogate electronics unit 130 constantly monitors the state of the sensors 140 to determine if a male user has penetrated the vaginal cavity 135. The electronics unit 130 measures the depth of penetration by comparing the pressure sensations experienced by adjacent sensors in a linear manner. The electronics unit 130 also monitors the amount of pressure exerted by the male user normal to the wall of the vaginal cavity.

These measurements enable the electronics unit 130 to create contact messages for depth of penetration and contact pressure. These contact messages are then conveyed to the first user computer 15 by a serial interface 20. A communications network 25 is used to convey the contact messages from the first user computer 15 to the second user computer 30 and then through to the male surrogate 35.

The female surrogate 10 may further comprise a moisture sensor 150 disposed at the end of the vaginal cavity 135. When the male user ejaculates within the vaginal cavity 135, the moisture sensor 150 detects the presence of the male user's seminal fluid. The female surrogate electronics unit 130 monitors the moisture sensor 150. When the moisture sensor 150 becomes active, the electronics unit 130 will create a contact message that indicates the presence of the male user seminal fluid. This contact message is then conveyed to the male surrogate 35 using the communications path already described.

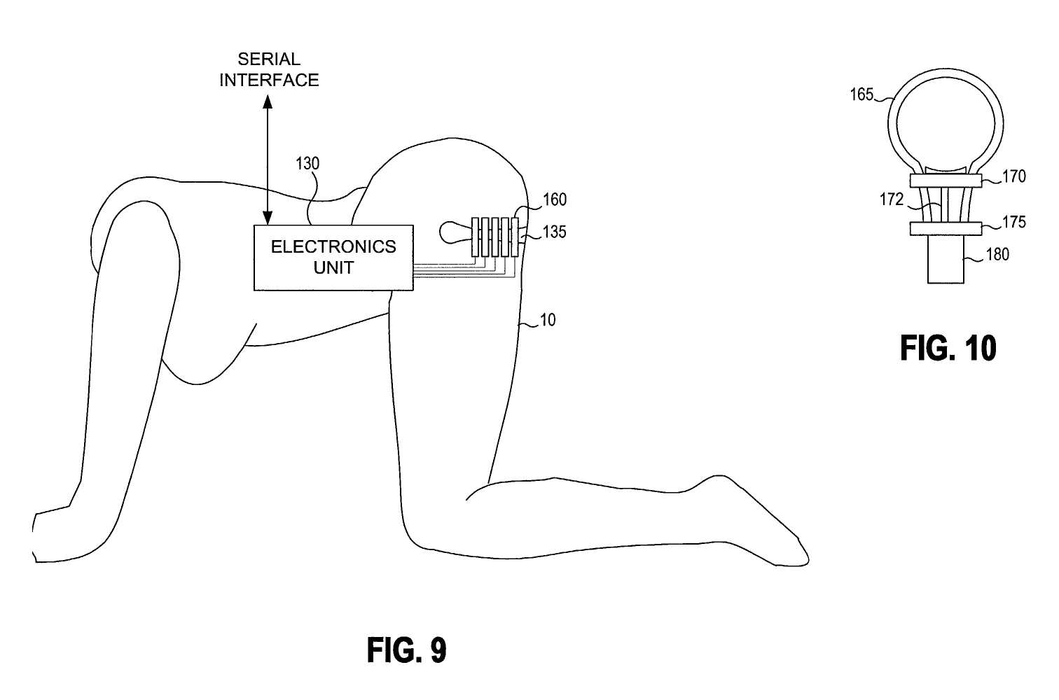

FIG. 9 is a pictorial diagram that depicts the placement of constriction actuators within the female surrogate. In order to convey a sensation of pressure to the male user that mimics the pressure exerted by a female user upon the male surrogate 35, constriction actuators 160 are placed concentrically to the vaginal cavity 135. Contact messages received by the female surrogate 10 can be interpreted by the electronics unit 130 so that the constriction actuators 160 can be commanded to mimic the pressure imparted on the male surrogate 35.

FIG. 10 is a detailed view of a constriction actuator according to the present invention. Each constriction actuators is comprised of a constriction ring 165, a pressure block 170, a plunger 172, a stop block 175 and a linear actuator 180.

The constriction ring 165 is length of flexible material approximately ½ inch in width attached at either end to the stop block 175. The pressure block 170 contains two slots through which the constriction ring 165 is routed through. Upon command from the electronics unit 130, the linear actuator 180 exerts force upon the pressure block 170. As the pressure block moves away from the stop block 175, the constriction ring 165 is drawn through the two slots in the pressure block 170. This results in a decrease in the diameter of the constriction ring 165 that ultimately imparts radial pressure on the male user's penis.

Alternative Embodiments

While this invention has been described in terms of several preferred embodiments, it is contemplated that alternatives, modifications, permutations, and equivalents thereof will become apparent to those skilled in the art upon a reading of the specification and study of the drawings. It is therefore intended that the true spirit and scope of the present invention include all such alternatives, modifications, permutations, and equivalents. Some, but by no means all of the possible alternatives are described herein.

1. A female surrogate comprising:
vaginal cavity having a diameter and an inner surface;
plurality of sensors disposed at the inner surface of the vaginal cavity for detecting the depth of penetration of an object into the vaginal cavity;
electronics unit that receives information from the plurality of sensors and creates contact messages that represent the amount of penetration of an object into the vaginal cavity; and
interface that the electronics unit uses to convey contact messages to an external device.

2. The female surrogate of claim 1 wherein the plurality of sensors are pressure sensors.

3. The female surrogate of claim 1 wherein the plurality of sensors are strain gages.

4. The female surrogate of claim 1 further comprising:
plurality of actuators for constricting the diameter of the vaginal cavity and
wherein the electronics unit receives contact messages arriving by the interface that describe the amount of force that the inner surface of the vaginal cavity must exert on an object inserted into the vaginal cavity and wherein the electronics unit commands the plurality of actuators to constrict the diameter of the vaginal cavity so as to impose a force upon an object inserted into the vaginal cavity.

5. The female surrogate of claim 1 wherein the plurality of actuators comprise:
constriction ring having a first end and a second end;
pressure block having slots that accept the first and second ends of the constriction ring;
stop block to which the first and second ends of the constriction ring are attached; and
linear actuator that exerts force upon the pressure block so as to draw the first and second ends of the constriction ring toward the stop block relative to the pressure block.

6. The female surrogate of claim 1 further comprising:
moisture sensor that detects moisture in the vaginal cavity and
wherein the electronics unit monitors the state of the moisture sensors, creates a contact message indicating that the moisture sensor has detected moisture and conveys the contact message to an external device using the interface.

7. A male surrogate comprising:
housing;
artificial penis protruding from the housing having an external surface and a tip;
extension mechanism that causes the artificial penis to extend outward from the housing;
interface that receives contact messages; and
electronics unit that receives contact messages using the interface that describe the amount of penetration that the artificial penis must produce and commands the extension mechanism to extend the artificial penis in accordance with said contact message.

8. The male surrogate of claim 7 wherein the extension mechanism comprises a pulley driven by a stepper motor having a heavy gage wire attached to the pulley at one end and to the artificial penis at the other end.

9. The male surrogate of claim 7 wherein the extension mechanism comprises a pulley driven by a servomotor having a heavy gage wire attached to the pulley at one end and to the artificial penis at the other end.

10. The male surrogate of claim 7 further comprising:
plurality of sensors disposed on the outer surface of the artificial penis and wherein the electronics unit monitors the status of the sensors to determine the amount of pressure exerted upon the artificial penis and creates contact messages that are sent to external devices using the interface that depict the amount of pressure exerted upon the artificial penis.

11. The male surrogate of claim 7 further comprising:
sheath covering the outer surface of the artificial penis to form an expansion cavity;
expansion-liquid reservoir for containing a quantity of expansion-liquid; and
pump that receives expansion-liquid from the reservoir and delivers it to the expansion cavity wherein the electronics unit receives contact messages by means of the interface that depict the amount of pressure experienced by a female surrogate and commands the pump to force thickened liquid into the expansion cavity to replicate the pressure indicated in the contact message.

12. The male surrogate of claim 7 further comprising:
ejaculant reservoir for containing a quantity of ejaculant liquid;
ejaculant pump that receives ejaculant liquid from the ejaculant reservoir; and
catheter having a first end connected to the ejaculant pump so as to receive ejaculant liquid from the ejaculant pump and a second end having an opening that terminates at the tip of the artificial penis and
wherein the electronics unit receives contact messages using the interface that depict the need to dispense ejaculant and commands the ejaculant pump to dispense ejaculant in accordance with the contact message.

13. A system for remote sexual contact comprising:
female surrogate having a vaginal cavity that creates contact message indicative of the physical contact imparted upon the vaginal cavity;
first user computer that receives contact messages from the female surrogate and conveys to the contact messages to a communications network;
male surrogate having housing and an artificial penis that protrudes from the housing in response to contact messages; and
second user computer that receives contact messages from a communications network and delivers them to the male surrogate.

14. A method for remote sexual contact comprising the steps of:
monitoring sensors disposed in a female surrogate in order to determine the type of physical contact imparted on the female surrogate by a user;
creating contact messages that describe the contact perceived by the sensors;
conveying the contact messages to a male surrogate; and
causing the male surrogate to mimic the contact described in the contact messages.
